(19) United States
(12) Patent Application Publication
(10) Pub. No.: US 2004/0161366 A1
(43) Pub. Date: Aug. 19, 2004

(54) Devices, tests and methods for the analysis of wine samples

(76) Inventor: Michael M. Miller, Corvallis, OR (US)

Correspondence Address: LORI M FRIEDMAN, 300 BENTON VIEW DRIVE, PHILOMATH, OR, 97370, US

(21) Appl. No.: 10/775,691

(22) Filed: Feb. 10, 2004

Related U.S. Application Data
(63) Continuation-in-part of application No. 09/695,688, filed on Oct. 25, 2000, now abandoned.

(51) Int. Cl. G01N 33/14

(52) U.S. Cl. 422/58; 436/24

(57) ABSTRACT

The present invention provides a method for analyzing wine samples using a multi-layered test strip. The test strip is part of a kit that includes a plurality of the strips, a plurality of samplers, and a plurality color charts and written explanations of the test results which test kit components produce values for the determination of malic acid, lactic acid, residual yeast-fermentable sugar, and pH.

### RELATED PATENT APPLICATIONS

This is a continuation in part utility patent application based on U.S. patent application Ser. No. 09/695,688 filed Oct. 25, 2000.

### FIELD OF THE INVENTION

The present invention relates to the calorimetric analysis of wine. Provided are methods, a test kit, and procedures for testing for pH, lactic acid, malic acid, and residual yeast-fermentable sugar content of wine samples. More specifically, the tests and methods are designed for accurate calorimetric determination of wine due to the multi-level test pads which include a heretofore unknown interference removal layer for isolation of the anthocyanin -based color.

### BACKGROUND AND RELATED ART

The wine making process involves the maturation and aging of fermenting grapes and involves a series of changes that lead to a wine with optimal appearance, color, taste and flavor. The color of red wines is a result of anthocyanin pigments in various amounts and forms. Anthocyanins and related compounds have received the attention of food chemists because of their importance to the color quality of fruits and vegetables.

There are many known methods for measuring and reporting anthocyanin pigment content in food and beverage products Many systematic methods for identification of anthocyanin pigments are well established, and more than 300 anthocyanins have been identified in nature, involving analytical methods and sophisticated equipment..

The present invention relates to a unitized dry reagent test device for the calorimetric determination of analytes and/or measurement of pH in samples containing tannin-based interfering substances, particularly anthocyanin. The methods and devices of the present invention are ideally suited for field or on-site testing, as well as in a laboratory.

In the testing of fruit juices or extracts of plants it is frequently necessary to remove colored tannins and anthocyanins in order to prevent the colored materials from interfering with an assay for an analyte in wine or other sample. In this invention, a multilayer test strip device is presented that is used in sampling wine. The test strip has several active layers for the colorimetric determination of wine samples. It comprises a polymeric, non-reactive support element with three distinct layers of varying lengths and an opening through which the reagent test pad can be viewed, in order

a) an adsorptive top layer of a non-woven fabric which facilitates wetting of a plurality of layers below it; said layers prepared in varying lengths wherein each layer is longer than the one below it and each is independently attached to the support element by adhesive;

b) an interference removal pad that exhibits adsorptive properties towards anthocyanin-based substances in wine;

c) a reagent test pad that comprises a small-porosity membrane.

The test strip's interference removal layer separates it from any other wine analysis test strips or kits, and the detailed description that follows includes an enabling disclosure of the constituents of the strip and procedures for its use.

There are other test strips available for wine sampling. Among these are the REFLECTOQUANT® system, made available by Merck KGaA, Darmstadt, Germany. The Reflectoquant system provides wine test strips as part of a system that uses remission photometry to analyze test strips that have been immersed in samples of analyte. The Reflectoquant system can analyze a variety of liquids, wine being one of many analytes. When analyzing wine samples, there is no provision for the removal of tannin-based interference elements such as anthocyanin.

On-device non-instrumental methods for removing the influence of interfering materials from test samples intended for use with reagent test strip devices are known, but none has proven appropriate or satisfactory for the removal of tannins, anthocyanins, and other colored and/or chemically interfering substances from food samples such as fruit juice and wine.

Carroll et al describe a diagnostic sanitary test strip in U.S. Pat. No. 6,040,195 which analyzes blood samples, including removal of interferences involved in the analysis of blood. In U.S. Pat. No. 5,304,468 Phillips et al reveal reagent test strips for determining blood glucose. In U.S. Pat. No. 5,178,831 Sakota et al uses a calorimetric device for testing body fluids. Performing analyses of human blood and other fluids is not useful when testing samples such as red wine. Blood has a broad absorption spectra at acidic pH in the range 500-550 nm, and at alkaline pH. The chemical reactions which take place in blood analysis are distinct from those used testing wine and other food samples. For example, the pH used for detection of some key wine analytes, the absorption maxima of tannins and anthocyanins shifts to the 550-690 nm range. Shifting of the wavelength of detection is not a suitable solution to the problem of colored substances interfering in the analysis of foods containing tannins and anthocyanins.

There are test strips employed for analytes other than blood or wine. For example, Test strips are for testing a variety of analytes are known. Among these are U.S. Pat. No. 4,223,089 to Rothe et al which details the analysis of ammonia with a multi-layer test strip. There is neither analysis of color impurities nor removal of said impurities in '089. Priest et al, in U.S. Pat. No. 5,824,491 uses a dry reagent test strip for measuring dissolved analyte in which chemical reactions, using benzidine dye precursors and antipyrene compounds, take place in reagent detection chemistry. Stephenson, in U.S. Pat. No. 5,601,061 has a description of screening and sampling of soil, wherein color changes indicate the amount of analyte present. The determining characterization matrix involves targeted porosity and density of solid samples. Colored interference members, and removal thereof for testing, are not part of '061.

Finally, Visiotti in U.S. Pat. No. 5,897,901 discloses a method for removing compounds that cause food discoloration in see-through packaging for fuit and vegetables. Even though food is a subject of this disclosure, the discoloration involved packaging materials therefor. The packaging is a multi-layer solid, and changes to it are distinct from the instant invention.

### BRIEF SUMMARY OF THE INVENTION

The present invention provides a method for analyzing wine samples using a multi-layered test strip. The test strip is part of a kit that includes a plurality of the strips, a plurality of samplers, and a plurality color charts and written explanations of the test results which test kit components produce values for the determination of malic acid, lactic acid, residual yeast-fermentable sugar, and pH.

One of the factors that differentiates this invention from any other test strip devices is that one of the three distinct layers of the test pad is an interference removal pad. The interference removal pad exhibits adsorptive properties towards anthocyanin-based substances in wine. More specifically, the interference removal pad is selected from the group consisting of polyamides and membranes produced from unmodified amphoteric nylon 6,6, and possessing an equivalent number of amino and carboxyl end groups and having a porosity ranging from about 0.1 to 0.45 microns.

Further unique features of the devices, tests and methods for the analysis of wine samples will be made clear with the figure and detailed description that follow.

### BRIEF DESCRIPTION OF THE DRAWING

FIG. 1 is a description of the test kit of this invention. It shows the detailed structure of the multi-layer testing device of the invention and a representation of the sampling means, color charts, and explanatory instructions included in the kit of this invention.

### DETAILED DESCRIPTION OF THE PREFERRED EMBODIMENTS

The present invention describes devices, tests and methods for the analysis of wine samples. In this invention, the word “wine” will be used to mean the colored, fermented juice of grapes used as a drink. It will also refer to “must”, which in this invention will mean the juice of freshly crushed grapes that will be fermented into wine. Must can include pulp, skins, and seeds. It is the combination of grapes, juice, and skins that ferment to create wine.

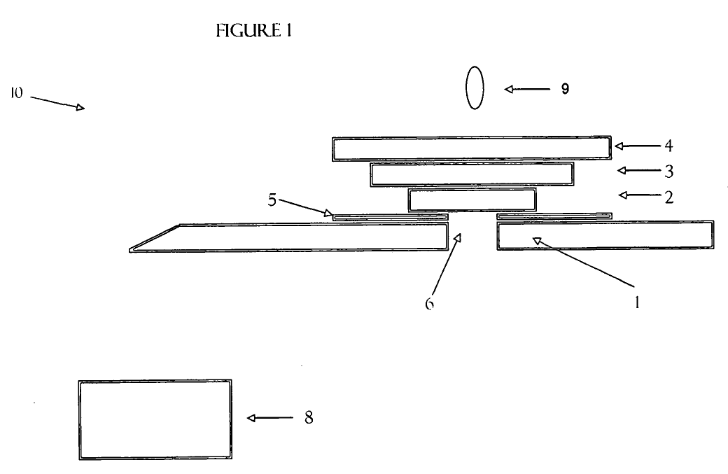

The test strip 10, as shown in FIG. 1, comprises a structure with a polymeric, non-reactive support element 1 with three distinct layers of varying lengths. At the top of the test strip 10 an adsorptive top layer of a non-woven fabric 4 which facilitates wetting of a plurality of layers below it; said layers prepared in varying lengths wherein each layer is longer than the one below it and each is independently attached to the support element 1 by adhesive 5. Below the adsorptive top layer 4 is an interference removal pad 3 that exhibits adsorptive properties towards anthocyanin-based substances in wine. Below the interference removal pad 3 is the reagent test pad 2 that comprises a small-porosity membrane.

There is an opening 6 through which the reagent test pad 2 can be viewed, The person viewing the reagent test pad 2 can compare the color seen to the color of a chart of color standards When being tested by the kit of this invention, the wine sample is dropped onto the adsorptive top layer 4 after which it proceeds downward as was just described. The tests that are performed on the wine sample are selected from the group consisting of pH, lactic acid, malic acid, and residual yeast-fermentable sugar.

For all the tests that are performed on wine samples using the present invention, a variation of the following basic procedure is done

A 10-20 micro-liter sample of wine is taken from the sample of interest with a sampling device 8. The sample is then dropped on an adsorptive layer 4 on top of a test strip 10 after which it is proceeds to the interference removal pad 3. The droplet then proceeds to the reagent test pad 2 where it reacts with at least one colorimetric indictor.

After the droplet is applied to the adsorptive top pad 4, the user waits from one to ten minutes for color development. When the color appears, it can be viewed by looking at the opening 6 which is part of the pre-scored and pre-punched non-reactive support 1. The user can then compare the color developed by the wine sample 9 to that of a standard color chart (not shown) that is part of the kit of this invention to assay the wine source from which the sample 9 was taken. After corrections are made for any dilutions that were made to the sample 9, the wine source can be maximized for quality and adjustments to the wines pH, lactic acid content, malic acid content, residual yeast-fermentable sugar and the like may be made as desired.

It is noteworthy that the test as performed in this manner needs no analytical instrument and uses just the eye to judge the color match achieved by the wine drop to established standards. The test strips 10 are disposable, easy to use, and accurate. This is a significant difference from prior art and commercially available test strips which require instrumental, calorimetric analysis and do not have an interference removal pad or any other means for removing the anthocyanin-based interference moieties from the wine sample 9.

### Specific Test Procedures

The kit of the present invention allows a user, who may be a home wine producer, a commercial winery, an individual interested in learning more about a certain wine, or anyone interested in the analysis of wine. Tests that have been developed and are supplied with the kit (available and sold commercially by ACCUVIN) include pH, lactic acid content, malic acid content, residual yeast-fermentable sugar. Other tests which are thought to be possible but are not yet commercially available by Accuvin include acetaldehyde, citric acid, potassium, alcohol, titratable acidity, harvest sugar, carbon dioxide, tannins and sulfur dioxide.

1, Lactic Acid Test Procedure: Analysis of a wine sample to measure the lactic acid level of wine that is undergoing malolactic fermentation lactic acid includes the steps of

i) diluting the wine sample in a ratio of 1:10 if necessary to keep the lactic acid concentration of the test sample in the range of from zero to 400 mg/L;

ii) squeezing a sampler bulb;

iii) dipping the tip of the sampler tip into wine;

iv) releasing the bulb to aspirate sample;

v) transferring the sample to an adsorptive layer on the back of a test strip by squeezing the sampler bulb;

vi) allowing a sample droplet to absorb into adsorptive layer after which the sample proceeds downward through an interference removal pad, then continues to a reagent test pad where it reacts with at least one calorimetric indicating material;

vii) waiting about two minutes for color development;

viii) viewing the color developed;

ix) determining the lactic acid level by comparing the developed color to a standard color chart provided, making corrections to compensate for any dilution that was done;

x) maximizing the quality of wine by inoculating for malolactic fermentation if lactic acid levels are low and malolactic fermentation is desired.

2. Malic Acid Test Procedure: Analysis of a wine sample to measure the malic acid level of wine that is undergoing malolactic fermentation lactic acid includes the steps of

i) diluting the wine sample in a ratio of 1:20 if necessary to keep the malic acid concentration of the test sample in the range of zero to 500 mg/L;

ii) squeezing a sampler bulb;

iii) dipping the tip of the sampler tip into wine;

iv) releasing the bulb to aspirate sample;

v) transferring the sample to an adsorptive layer on the back of a test strip by squeezing the sampler bulb;

vi) allowing a sample droplet to absorb into adsorptive layer after which the sample proceeds downward through an interference removal pad, then continues to a reagent test pad where it reacts with at least one calorimetric indicating material;

vii) waiting about four minutes for color development;

viii) viewing the color developed;

ix) determining the malic acid level by comparing the developed color to a standard color chart provided, making corrections to compensate for any dilution that was done;

x) maximizing the quality of wine if low levels of malic acid are indicated by the developed color by adding preservative levels of sulfur dioxide to prevent the growth of undesired bacteria.

3. Test Procedure for Measuring a wine's residual yeast-fermentable sugar in a fermented wine: includes the steps of

i) diluting the wine sample in a ratio of 1:20 if necessary to keep the concentration of the test sample in the range of from zero to 2000 mg/L;

ii) squeezing a sampler bulb;

iii) dipping the tip of the sampler tip into wine;

iv) releasing the bulb to aspirate sample;

v) transferring the sample to an adsorptive layer on the back of a test strip by squeezing the sampler bulb;

vi) allowing a sample droplet to absorb into adsorptive layer after which the sample proceeds downward through an interference removal pad, then continues to a reagent test pad where it reacts with at least one colorimetric indicating material;

vii) waiting about two minutes for color development;

viii) viewing the color developed;

ix) determining the residual yeast-fermentable sugar level by comparing the developed color to a standard color chart provided, making corrections to compensate for any dilution that was done;

x) maximizing the quality of wine by adding preservative levels of sulfur dioxide as soon as the yeast-fermentable sugar concentration has reached desired levels as indicated by the developed color.

4. Test Procedure for measuring the pH of a wine sample including the steps of

i) squeezing a sampler bulb;

ii) dipping the tip of the sampler tip into wine;

iii) releasing the bulb to aspirate sample;

iv) transferring the sample to an adsorptive layer on back of a test strip by squeezing the sampler bulb;

v) allowing a sample droplet to absorb into adsorptive layer after which the sample proceeds downward through an interference removal pad, then continues to a reagent test pad where it interacts with at least one colorimetric indicating material;

vi) waiting about three minutes for color development;

vii) determining the pH by comparing the developed color to a standard color chart provided.

vii. maximizing wine quality by ensuring wine is at the pH level for optimum flavor as indicated by the developed color, or by adjusting pH levels until the desired range has been attained.

5. Details of the Characterization of the Test Strip 10: The test strip 10 provides for the calorimetric determination of wine samples. It is comprised of a polymeric, non-reactive support element 1 with three distinct layers of varying lengths and an opening 6 through which the reagent test pad can be viewed.

a) The adsorptive top layer 4 of a non-woven fabric facilitates wetting of a plurality of layers below it; said layers prepared in varying lengths wherein each layer is longer than the one below it and each is independently attached to the support element by adhesive. In this invention it is made by Dupont, Asturias, Spain under the name Sontara, style 9951.

b) The interference removal pad 3 that exhibits adsorptive properties towards anthocyanin-based substances in wine was discovered by applicant through experimentation, the details of which are described below: Experiments were conducted to find suitable materials for the interference removal pad 3 of this invention. The results of the experimentation are tabulated below:

| | Visual | |
|---|---|---|
| Interference Removal Pad Material | Rating | Reflectance |
| Ion exchange resin-impreg. Paper (Whatman) | 2 | 0.030 |
| Activated carbon-impreg. Paper (S&S) | 0 | 0.000 |
| Polyamide membrane (Cuno) | 4 | 0.190 |
| Polyamide membrane (S&S) | 0 | 0.020 |
| Quantitative filter paper (Whatman) | 3 | 0.130 |
| Polysulfone membrane (Pall) | 4 | n/a |
| Polyamide membrane (Pall) | 0 | 0.010 |

From these data it can be seen that polyamide membranes and activated carbon-impregnated paper membranes are effective at removing anthocyanins and other colored interfering substances from test samples. Preferred embodiments for the interference removal pad 3 are selected from the group consisting of polyamides and membranes produced from unmodified amphoteric nylon 6,6, and possessing an equivalent number of amino and carboxyl end groups and having a porosity ranging from about 0.1 to 0.45 microns.

c) the preferred reagent test pad 2 was likewise discovered experimentally by applicant. The preferred materials for the reagent test pad 2 are selected from the group consisting of polysulfones, polyamides, and filter paper.

6. Kit parts from various commercial suppliers:

A preferred interference removal pad 3 is Biodyne® polyamide membrane, available from Pall Corp. The reagent test pad 2 is a polyamide membrane available from Cuno, Meriden, Conn., which was impregnated with an aqueous solution comprising approximately 1.32 g/L bromphenol blue, 1.0 g/L Triton X-100 and 2.0 g/L gelatin. Following impregnation excess fluid was blotted off and the reagent test pad 2 air dried for at least 30 min. prior to use. Reagent test pads 2 were 0.25 inch diameter circles. The interference removal pads were 5/16 inch squares. The preferred adsorptive pad 4 of the test strip 10 is approximately 5/16 inches by 7/16 inches in size, and is wetted with 15-20 microliters of liquid. The liquid being tested migrates through the interference removal pad 3 in which interfering substances are trapped. Target analytes migrate to the reagent test pad 2 where appropriate chemical reactions occur. Any color change in the reagent test pad was observed through the viewing opening 6.

The sampling means 8 that are part of the test kit of this invention are supplied by the Samco Scientific Corporation of San Fernando, Calif. They are single-use disposable pipettes and a plurality of them are supplied with the test kit of this invention that is sold.

### SCOPE OF THE INVENTION

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiments disclosed. On the contrary, the intention is to cover all modifications and alternate constructions coming within the spirit and scope of the invention as generally expressed by the following claims, which particularly point out and distinctly claim the subject matter of the invention:

What is claimed is:

1. A method for analyzing wine in a series of steps comprising
a) removing a ten to twenty micro-liter test sample of wine from a container of wine using a sampling device;
b) allowing the sample droplet to absorb into an adsorptive layer of a test strip after which the sample proceeds downward through an interference removal pad, then continues to a reagent test pad where it reacts with at least one calorimetric indicating material;
c) waiting from one to ten minutes for color development;
d viewing the color that has developed by looking at a hole provided in a polymeric, non-reactive support that is pre-punched and pre-scored;
e) comparing the developed color to a standard color chart provided, making corrections to compensate for any dilution that was done;
f) using the color comparison chart to assay the wine source from which the sample was taken;
g) adjusting the wine source as desired for maximizing quality as indicated by color.

2. The method of claim 1 wherein analysis of the wine sample is for measuring the lactic acid level of wine that is undergoing malolactic fermentation lactic acid which includes the steps of
i) diluting the wine sample in a ratio of 1:10 if necessary to keep the lactic acid concentration of the test sample in the range of from zero to 400 mg/L;
ii) squeezing a sampler bulb;
iii) dipping the tip of the sampler tip into wine;
iv) releasing the bulb to aspirate sample;
v) transferring the sample to an adsorptive layer on the back of a test strip by squeezing the sampler bulb;
vi) allowing a sample droplet to absorb into adsorptive layer after which the sample proceeds downward through an interference removal pad, then continues to a reagent test pad where it reacts with at least one calorimetric indicating material;
vii) waiting about two minutes for color development;
viii) viewing the color developed;
ix) determining the lactic acid level by comparing the developed color to a standard color chart provided, making corrections to compensate for any dilution that was done;
x) maximizing the quality of wine by inoculating for malolactic fermentation if lactic acid levels are low and malolactic fermentation is desired.

3. The method of claim 1 wherein the analysis of the wine sample is for measuring the malic acid level of wine that is undergoing malolactic fermentation which includes the steps of
i) diluting the wine sample in a ratio of 1:20 if necessary to keep the malic acid concentration of the test sample in the range of zero to 500 mg/L;
ii) squeezing a sampler bulb;
iii) dipping the tip of the sampler tip into wine;
iv) releasing the bulb to aspirate sample;
v) transferring the sample to an adsorptive layer on the back of a test strip by squeezing the sampler bulb;
vi) allowing a sample droplet to absorb into adsorptive layer after which the sample proceeds downward through an interference removal pad, then continues to a reagent test pad where it reacts with at least one calorimetric indicating material;
vii) waiting about four minutes for color development;
viii) viewing the color developed;
ix) determining the malic acid level by comparing the developed color to a standard color chart provided, making corrections to compensate for any dilution that was done;
x) maximizing the quality of wine if low levels of malic acid are indicated by the developed color by adding preservative levels of sulfur dioxide to prevent the growth of undesired bacteria.

4. The method of claim 1 wherein the analysis of the wine sample is for measuring the levels of residual yeast-fermentable sugar in wine including the steps of
i) diluting the wine sample in a ratio of 1:20 if necessary to keep the concentration of the test sample in the range of from zero to 2000 mg/L;
ii) squeezing a sampler bulb;
iii) dipping the tip of the sampler tip into wine;
iv) releasing the bulb to aspirate sample;
v) transferring the sample to an adsorptive layer on the back of a test strip by squeezing the sampler bulb;
vi) allowing a sample droplet to absorb into adsorptive layer after which the sample proceeds downward through an interference removal pad, then continues to a reagent test pad where it reacts with at least one calorimetric indicating material;
vii) waiting about two minutes for color development;
viii) viewing the color developed;
ix) determining the residual yeast-fermentable sugar level by comparing the developed color to a standard color chart provided, making corrections to compensate for any dilution that was done;
x) maximizing the quality of wine by adding preservative levels of sulfur dioxide as soon as the yeast-fermentable sugar concentration has reached desired levels as indicated by the developed color.

5. The method of claim 1 wherein the analysis of the wine sample of for the measuring of pH including the steps of
i) squeezing a sampler bulb;
ii) dipping the tip of the sampler tip into wine;
iii) releasing the bulb to aspirate sample;
iv) transferring the sample to an adsorptive layer on back of a test strip by squeezing the sampler bulb;
v) allowing a sample droplet to absorb into adsorptive layer after which the sample proceeds downward through an interference removal pad, then continues to a reagent test pad where it interacts with at least one colorimetric indicating material;
vi) waiting about three minutes for color development;
vii) determining the pH by comparing the developed color to a standard color chart provided.
vii. maximizing wine quality by ensuring wine is at the pH level for optimum flavor as indicated by the developed color, or by adjusting pH levels until the desired range has been attained.

6. The method of claim 1 wherein the observation of the developed color is performed by comparing the developed color with that of a standard color chart and without the need for instrumental color intensity measurement

7. A testing device for the calorimetric determination of wine samples comprising a polymeric, non-reactive support element with three distinct layers of varying lengths and an opening through which the reagent test pad can be viewed, in order
a) an adsorptive top layer of a non-woven fabric which facilitates wetting of a plurality of layers below it; said layers prepared in varying lengths wherein each layer is longer than the one below it and each is independently attached to the support element by adhesive;
b) a reagent test pad that comprises a small-porosity membrane
c) an interference removal pad that exhibits adsorptive properties towards anthocyanin-based substances in wine.

8. The device of claim 7 wherein the reagent test pad is comprised of materials selected from the group consisting of polysulfones, polyamides, and filter paper.

9. The device of claim 7 wherein the interference removal pad is selected from the group consisting of polyamides and membranes produced from unmodified amphoteric nylon 6,6, and possessing an equivalent number of amino and carboxyl end groups and having a porosity ranging from about 0.1 to 0.45 microns.

10. The device of claim 7 wherein the wine samples are tested for substances selected from the group consisting of pH, malic acid, lactic acid, residual yeast-fermentable sugar, acetaldehyde, acetic acid, ammonia, citric acid, hydrogen sulfide, potassium, alcohol, titratable acidity, harvest sugar, amino nitrogen, carbon dioxide, tannins and sulfur dioxide.

11. The device of claim 7 wherein the wine sample is first deposited by droplets onto the top adsorptive layer, after which it proceeds to the next layer which is the interference removal pad whereby anthocyanin-based substances are trapped, after which it proceeds to the next layer which is the reagent test pad where colorimetric chemical reactions occur after a predetermined time period indicating the amount of lactic acid, malic acid, residual yeast-fermentable sugar, and pH of the sample.

12. A test kit for the calorimetric determination of wine samples comprising
a) a plurality of multi-layer disposable test strips;,
b) a plurality of disposable sampler devices,
c) a plurality of instructions for use;
d) a plurality color charts and written explanations of the test results which test kit components produce values for the determination of tests selected from the group consisting of malic acid, lactic acid, residual yeast-fermentable sugar, and pH.

13. The test kit of claim 12 wherein the multi-layer disposable test strip is comprised of a polymeric, non-reactive support element with three distinct layers of varying lengths and an opening through which the reagent test pad can be viewed;
a) an adsorptive top layer of a non-woven fabric which facilitates wetting of a plurality of layers below it, said layers made in varying lengths wherein each layer is longer than the one below it and each is independently attached to the support element by adhesive.
b) a reagent test pad that comprises a small-porosity membrane; and
c) an interference removal pad that exhibits adsorptive properties towards anthocyanin-based substances in wine.

14. The test kit of claim 12 wherein the sampler devices are disposable pipettes with volume ranging from about 20 to about 250 microliters.

15. The test kit of claim 13 wherein the interference removal pad is comprised of materials selected from the group consisting of membranes produced from unmodified amphoteric nylon 6,6, and possessing an equivalent number of amino and carboxyl end groups and having a porosity ranging from about 0.1 to 0.45 microns.

16. The test kit of claim 15 wherein the an interference removal pad that exhibits adsorptive properties towards anthocyanin-based substances in wine;

17. The test kit of claim 12 wherein the data analyzed are selected from the group consisting of pH, malic acid level, lactic acid level, titratable acidity, harvest sugar, residual yeast-fermentable sugar, sulfur dioxide, ammonia, amino nitrogen, target alcohol levels, acetic acid, carbon dioxide, hydrogen sulfide, acetaldehyde, citric acid, and tannins.
